(19) United States
(12) Patent Application Publication
(10) Pub. No.: US 2012/0191211 A1
(43) Pub. Date: Jul. 26, 2012

(54) Foot and ankle implant and associated method

(76) Inventor: Tim Drozd, Portland, OR (US)

(21) Appl. No.: 13/353,994

(22) Filed: Jan. 19, 2012

Related U.S. Application Data
(60) Provisional application No. 61/434,773, filed on Jan. 20, 2011.

(51) Int. Cl. A61F 2/42

(52) U.S. Cl. 623/21.18

(57) ABSTRACT

The present invention contemplates an implant for insertion in various bones of the human foot and ankle to repair injuries and deformities. In one preferred embodiment the implant device includes a trapezoidal body member when viewed from the top with substantially vertical sidewalls consisting of at least two intersecting sidewalls that define a head end having a head-height. The sidewalls diverge at their respective tail ends to define a concave shape and a tail-height that is substantially the same as the head height, thus giving the implant a top and bottom that are substantially parallel when viewed from the front, side, or back views. The top and bottom portions further include friction-enhancing serrations.

### PRIORITY CLAIM

The present application claims benefit under 35 USC Section 119(e) of U.S. Provisional Patent Application Ser. No. 61/434,773 filed on 2011-01-20. The present application is based on and claims priority from this application, the disclosure of which is hereby expressly incorporated herein by reference.

### BACKGROUND

The present invention relates to implantable devices to provide structural manipulation of various bones of the foot and ankle to correct abnormalities and deformities independent of shape of the implant to encourage anatomical healing.

The current state of the art includes various surgical procedures and prosthetic devices that attempt to correct foot and ankle disorders and deformities. Current reconstructive procedures include intra-operative shaping of autogenous bone tissue or human allograft bone tissue. Other bone grafting procedures include packing a void with a granular and/or putty-like material. Intra-operative shaping is a time-consuming process, and further the bone tissue used has limited size and shaping potential. The alternative of packing with granular and/or putty-like materials may not provide adequate structural support.

A more recent attempt to improve the state-of-the-art includes the device and method of Myerson et al. in Published U.S. Pat. App. No. 2007/0038303 published on 15 Feb. 2007 and filed on 15 Aug. 2006. Myerson teaches a method and anatomically shaped device for implantation between two bone portions of the foot or ankle to correct associated deformities. The implant device has a peripheral wall surrounding a central bore and defining an annular cross-section. The wall is constructed from a composite material that includes a ceramic component and a polymer component. The ceramic component is gradually resorbed after implantation, and the polymeric component gradually degrades after implantation. More specifically, the Myerson implant device comprises a composite structure having a ceramic component with macroporosity and a polymer component filling the macroporosity. The composite structure forms an anatomically shaped and load-bearing graft for implantation between two bone portions of the foot or ankle to correct associated deformities. The ceramic component is gradually resorbable after implantation, the polymeric component is gradually degradable after implantation and the composite structure is gradually replaceable by tissue/bone ingrowth. And, Myerson teaches a method for correcting foot/ankle deformities using this anatomically shaped composite implant device. The method includes providing a resorbable polymer-reinforced ceramic composite block, shaping the composite block to an anatomically-shaped and load-bearing graft for implantation between two bone portions of the foot or ankle to correct associated deformities, maintaining an opening between the two bone portions before inserting the implant, and inserting the implant in the opening such that the implant substantially matches the cross-section of the bone portions. Shaping of the composite block includes pre-operative or intra-operative shaping.

Myerson et al. builds upon a well-studied body of knowledge including methods and devices adapted for use in a Cotton osteotomy (a medial cuneiform plantarflexory opening wedge osteotomy). According to Daniel Yarmel, DPM, Gregory Mote, DPM, and Amber Treaster, DPM, AACFAX in “The Cotton Osteotomy: A technical Guide”, published in Vol. 48, No. 4 July/August 2009 in THE JOURNAL OF FOOT AND ANKLE SURGERY (p. 506-512)(the entire disclosure of which is incorporated herein by reference), use of an anatomically shaped wedge in medical cuneioform osteotomy to repair hallux abductovalgus has been known since at least as early as 1908, although the coined “Cotton” osteotomy dates to a 1936 article published by Frederic J. Cotton in THE NEW ENGLAND JOURNAL OF MEDICINE”. As Yarmel et al instruct, these early references clearly illustrate a wedge shaped graft or similar device and method wherein a small dorsal, longitudinal periosteal incision is made, and a periosteal elevator is used to expose the cortex of the medial cuneiform in preparation for osteotomy. Osteotomy is then made with the use of a sagittal saw, although osteotomes can also be used at the discretion of the surgeon. After completion of the bone cut, mobilization of the osteotomy can be achieved with the use of 2 small osteotomes, a single large osteotome or with the careful use of a smooth lamina spreader. Once the amount of cuneiform displacement and graft size are determined, placement of the subtalar joint in the neutral position as well as loading of the forefoot to simulate weightbearing are undertaken in an effort to assess the degree of deformity correction. Although the precise size and shape of the bone graft will vary in accordance with the requirements of the individual patient, the benefits of a dorsal cortical base are that it helps to prevent graft extrusion and collapse. A trapezoidal graft shape can also be used, typically measuring approximately 8 mm in the pediatric patient, and 4-6 mm in the adult. The use of a Weinraub Joint Spreader facilitates the insertion of the dorsally based graft.

Another representative state-of-the-art wedge system for foot and ankle surgery, the Biofoam brand wedge system available from Wright Medical Technology, Inc. of Arlington, Tenn., teaches a cancellous titanium matrix that mimics the cancellous bone structure when imaged at a magnification of 100×. The Biofoam matrix includes a porosity of up to 70% and mimics the strength and flexibility of a true cancellous bone, including a compressive modulus close to that of cancellous bone. The Biofoam wedge system teaches two unique shapes, specifically for use in an Evans Procedure for Lateral Column Lengthening or a Cotton Procedure for plantar flexion opening wedge osteotomies of the Medial Cuneiform. Further, this prior-art system teaches a surface plate or a screw targeted through the implant's central auxiliary hole, is required to ensure a stable environment for bone ingrowth. And, it further teaches that the use of a biologic may be added into the central auxiliary hole of the implant. Geometrically, this system appears wedge shaped in profile with a leading edge of a first height that is smaller than the trailing edge. From the top, the wedge appears either rectilinear with rounded corners and generally parallel running opposite side walls arranged at a generally 90-degree orientation relative to its adjacent sidewall, or in an alternative embodiment, ovoid with a trapezoidal appearance in the top view. In both embodiments the wedge includes a hollow center portion encapsulated by the sidewalls.

Yet other teachings in the current art instruct methods that eliminate a bone graft and, instead, utilize an Arthrex Low Profile Plate and Screw to correct for a high-end hallux valgus deformity. According to the teaching of Hardy et al. in their article entitled “Opening Base Wedge Osteotomy of the First Metatarsal Using the Arthrex Low Profile Plate and Screw System”, published in THE FOOT AND ANKLE ONLINE JOURNAL, Vol. 2, No. 4 (April 2009)—the entire disclosure is hereby incorporated by reference as if fully set-out herein—a titanium opening wedge plate with screws set a osteomized Metatarsal at a desired angle.

Although the existing procedures and implants for foot and ankle applications can be satisfactory for their intended purposes, there is still a need for implants that provide better structural support as well as size and shape versatility for various foot/ankle procedures. More specifically, there is a need for an implantable device that is independent of anatomical shape, yet the device needs to encourage anatomical healing that maximizes healed bone volume by minimizing the prevention or interruption of continuous bone-to-bone healing.

### SUMMARY OF THE INVENTION

The present invention contradicts the teaching of the current art and overcomes inherent limitations therein. The present invention contemplates a device, system, and method for correcting various foot and ankle disorders and deformities.

The present invention includes various embodiments of an implant device that is sized and shape independent of the target insertion anatomy. One advantage of the various embodiments of implant devices is that they are well suited to many different bones to correct many different disorders. The various embodiments of the contemplated invention are particularly well adapted for use in the foot and ankle of human patients, but would work equally well in other species and in other locations of the anatomy.

Advantages of the various preferred embodiments of the present invention include: A mechanical shape adapted to maximize healing that is not limited to the anatomical-shaped designs as taught in the prior-art; Constructed from a single material that has radioflucency or degrees of radiolucency; A material composition that is not resorbed nor degrades; in certain preferred embodiments a uni-sloped, rectangular (or alternately, trapezoidal) shape with limited radii to reduce stress risers stress risers with minimized cortical bone implant footprint area correctly proportionate to the cancellous footprint area of the implant providing an implant design which is more efficient and stable throughout all phases of healing which resists implant migration and/or rotational as well as torsion movement and thus allowing for maximized continuous and natural healed bone volume; Minimal wedge shape or reversed wedge shape to minimize expulsion; Elimination of cross bars to create a more efficient and stable design; Distinct transverse, frontal, and coronal planes; a design that maximizes healing and is independent of the bone cross section and requires less than two-thirds the bone depth; Not wedge shaped with divergent, unparalleled surfaces; and Non-porous. Of course, it should be understood that various combinations and sub-combinations of these elements are contemplated in other embodiments of the present invention.

### DRAWING

FIG. 1 is a top view of a first preferred embodiment of the implant device according to the present invention.

FIG. 2 is a right side view of the embodiment of FIG. 1.

FIG. 3 is a front view of the embodiment of FIG. 1.

FIG. 4 is a back view of the embodiment of FIG. 1.

FIG. 5 is a top view of a delta-shaped implant device according to a second preferred embodiment of the present invention

FIG. 6 is a top view of a flat-head shaped implant device according to a third preferred embodiment of the present invention.

FIG. 7 is a top view of a N-shaped implant device according to a fourth preferred embodiment of the present invention.

FIG. 8 is a detail and enlarged sectional view of FIG. 2.

FIG. 9 is a top view another embodiment of an implant device according to the present invention.

FIG. 10 is a top view of a 3-pointed implant device according to yet another embodiment of the present invention.

FIG. 11 is a top view of a 3-pointed implant device according to again another embodiment of the present invention.

FIG. 12 is a top view of a 3-pointed triangle implant device according to yet another embodiment of the present invention.

FIG. 13 is a top view of a 4-pointed “wishbone” shaped implant device according to yet another embodiment of the present invention.

FIG. 14 is a top view of an anchor-style three pointed implant device according to another embodiment of the present invention.

FIG. 15 is a top, cross-sectional view of a section of bone showing the orientation of a three-pointed implant device of the present invention.

FIG. 16 is a partial top view of one contemplated surface treatment for the implant device of the various embodiments of the present invention.

FIG. 17 is a partial top view of another contemplated surface treatment for the implant device of the various embodiments of the present invention.

FIG. 18 is a partial top view of yet another contemplated surface treatment for the implant device of the various embodiments of the present invention.

FIG. 19 is a top, cross-sectional view of a section of bone showing the orientation of an implant device of the present invention.

FIG. 20 is a top, cross-sectional view of a section of bone showing the orientation of yet another embodiment of the implant device of the present invention.

FIG. 21 is an offset frontal view of yet another embodiment of the present invention.

### DESCRIPTION OF THE INVENTION

Possible preferred embodiments will now be described with reference to the drawings and those skilled in the art will understand that alternative configurations and combinations of components may be substituted without subtracting from the invention. Also, in some figures certain components are omitted to more clearly illustrate the invention.

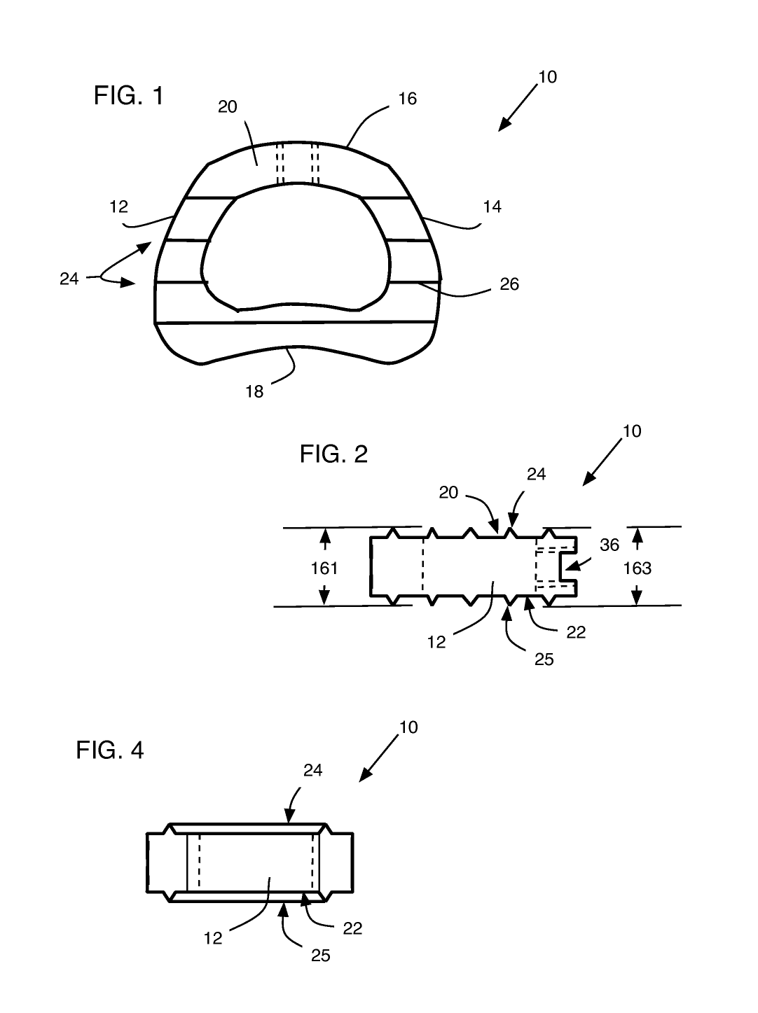

FIGS. 1 though 4 describe a first preferred embodiment of the present invention 1. This first preferred embodiment contemplates an orthopedic implant device for use to correct injuries and deformities of foot and ankle bones. The implant device resembles a rounded delta or trapezoidal shape when viewed from the top (as FIG. 1 illustrates) and consists of four arcuately shaped and interconnected adjacent sidewalls that rise substantially vertical from the top surface to the bottom surfaces.

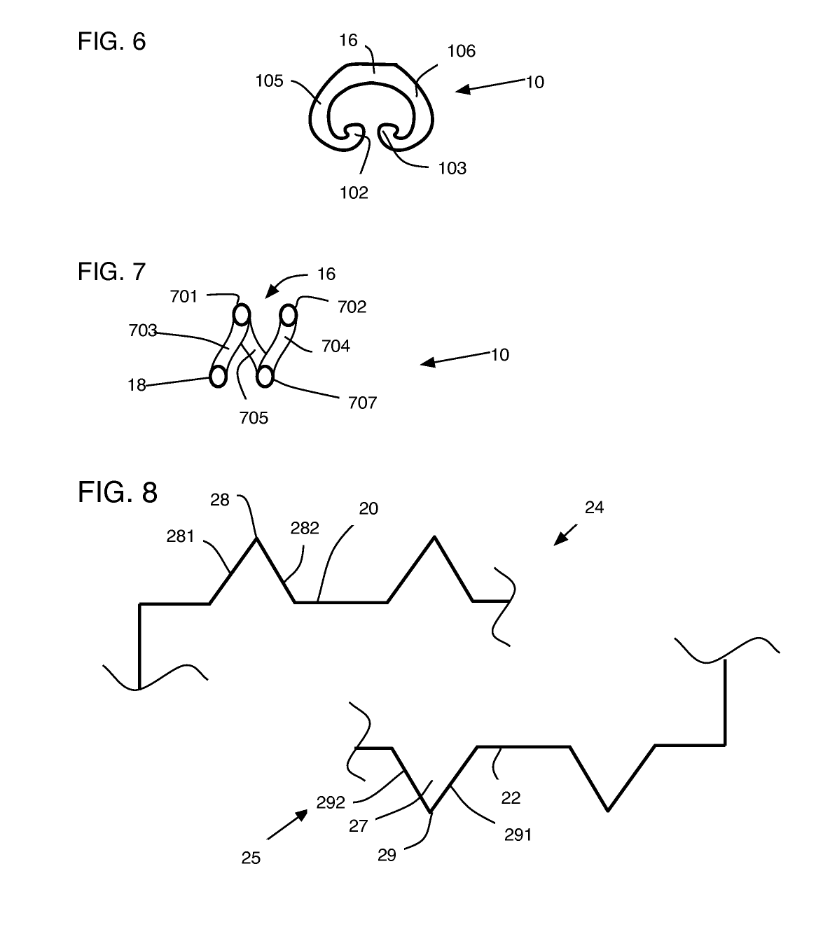

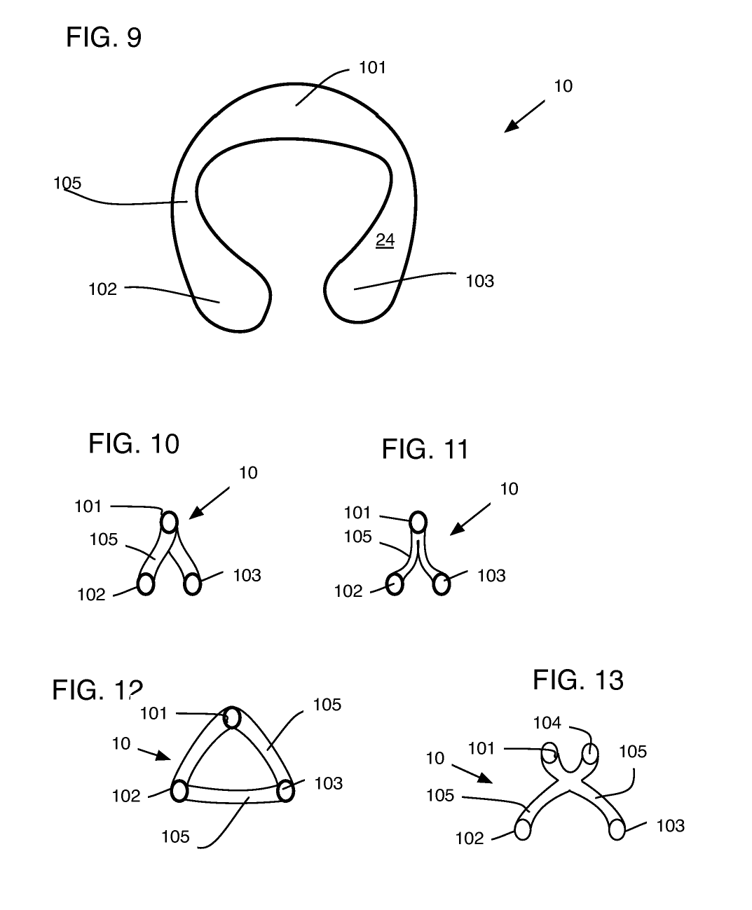

As FIGS. 1 though 7 show, the implant device includes at least two sidewalls, (FIG. 5), but also may include three sidewalls (FIG. 7), or the four sidewalls of FIGS. 1 and 6, for example. Thus, the implant device includes an implant body 10 comprising at least two side walls, the at least two sidewalls comprising a first sidewall 12 having a first-sidewall head-end 121 and a first-sidewall tail-end 123 and a second sidewall 14 having a second-sidewall head-end 141 and a second-sidewall tail-end 143; the implant body further comprising a head means 16 for joining at least two side walls at each respective head-end of each respective first and second sidewalls, the head having a first height 161 defined by a common height of the first-sidewall and second-side wall head-end, respectively. The implant body further having a second height 163, the second height being substantially the same as the first height, and the second height being defined by a common height of each respective first and second sidewalls at each respective tail-end 123 and tail-end 143. The implant body further comprising a top portion 20 arranged generally perpendicular to the at least two sidewalls and a bottom portion 22 disposed opposite the top portion and being generally parallel to the top portion; the top portion further including a top-friction enhancing feature 24 comprising of at least one top-serration 26 consisting of a top-peak 28 disposed at a first distance from the top and the top-peak further including a first 281 and second 282 support member arranged to intersect at a line defined by the top-peak and terminate adjacent to the top surface 20, the at least one top-serration being disposed generally parallel to any adjacent serrations, the at least one top-serration further arranged to extend horizontally across the top portion wherein an axis drawn from the head 16 to the tail 18 would define a vertical line; and the bottom portion further comprising a bottom-friction enhancing feature 25 comprising of at least one bottom-serration 27 consisting of a bottom-peak 29 disposed at a first distance from the bottom and the bottom-peak further including a first 291 and second support 292 member arranged to intersect at a line defined by the bottom-peak and terminate adjacent to the bottom surface, the at least one bottom-serration being disposed generally parallel to any adjacent serrations, the at least one bottom-serration further arranged to extend horizontally across the bottom portion.

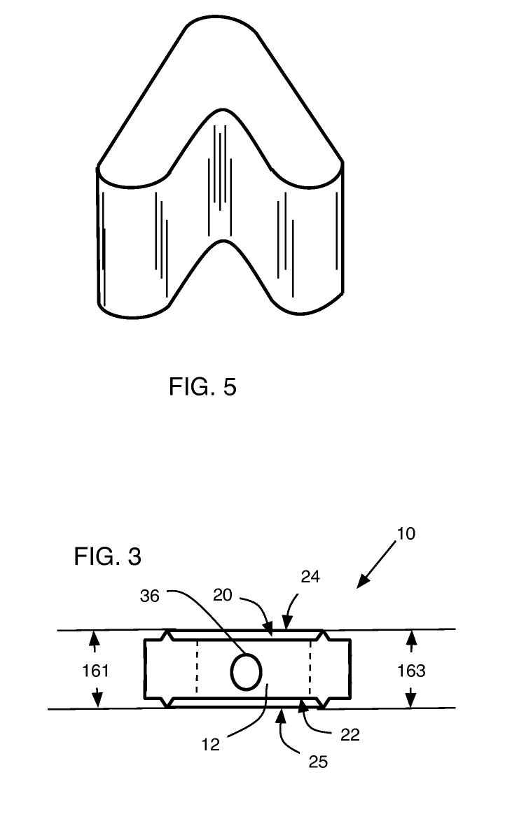

As FIGS. 1-4 illustrate, the implant device includes curvilinear sidewalls when viewed from the top and rectilinear when viewed from its respective profile. The first sidewall 12 comprises a first arcuate member 120 having a first acuate radius 122. The second sidewall 14 comprises a second arcuate member 140 having a second arcuate radius 142. And, in the first preferred embodiment the first and second arcuate radii are the same. Further, the first and second sidewalls cooperate to define a concave cavity therebetween.

Also, in the first preferred embodiment the head means 16 includes a vertical sidewall 160 having a third arcuate radius 162 defined by a member having a left head-end 164 and a right head-end 166 wherein the left head end couples to the first sidewall 12 and the right head-end couples to the second sidewall 14. And, the first and second sidewalls and head means define a concave cavity 34 therebetween. In an alternative embodiment it will be appreciated that the first sidewall 12, head sidewall 160, and second sidewall 14 can have identical radii and align on a common arc length, thus effectively replacing the three sidewalls with a single arcuate sidewall member extending from a left tail end to a right tail end and encompassing the first and second sidewalls and head sidewall. This arc-length radius can alter to be more narrow or more broad, as required for a particular use.

The implant device further includes a tail means 18, the tail means comprises a tail-member vertical sidewall 181 having a fourth arcuate radius 182 defined between the first sidewall tail-end 123 and the second sidewall tail end 143, the arcuate fourth sidewall arranged to have a convex face 183 disposed toward the concave cavity and a concave face 184 disposed opposite its convex face.

The tail 18 further includes a though hole 36 arranged on its concave face and extending through the tail-member sidewall terminating at the convex face. The hole 36 is adapted to slideably and selectively receive a bone screw, pin, and the like.

In the first preferred embodiment, both the top portion 20 and bottom portion 22 include a plurality of parallel serrations. These serrations, or top and bottom (respectively) friction enhancing features are disposed at a pre-determined spacing peak-to-peak.

In a second preferred embodiment, as FIG. 5 illustrates, the implant device of the present invention includes an implant body consisting of a first sidewall 12 consisting substantially of a first straight and vertical member 51, a second sidewall 14 comprising a second substantially straight and vertical member 52 and a head means 16 comprises an intersecting line 53 where the first sidewall head-end couples to the second sidewall head-end.

FIG. 6 illustrates a third preferred embodiment of an implant device contemplated by the present invention. Similar to the first preferred embodiment, this embodiment includes a head means 16 consisting of a pair of tail-end sidewalls with an opening in the back. The head means 16 comprises a substantially flat and vertical sidewall having a left head-end and a right head-end wherein the left head end couples to the first sidewall 105 and the right head-end couples to the second sidewall 106; and wherein the first and second sidewalls and head means define a concave cavity therebetween having an open portion at the tail of the implant, the tail having a left terminus 102 and a right terminus 103.

FIG. 7 illustrates a fourth preferred embodiment of the present invention 10 wherein three sidewalls are linked to form an N-shape and the head means 16 consists of leading ends 701 and 702 of the two outside sidewalls 703 and 704 with the third sidewall 705 linking the two outside sidewalls diagonally. Accordingly, the implant body 10 includes at least two sidewalls but, more specifically, three sidewalls. And, the three sidewalls arrange in a N-formation wherein the first sidewall head-end 16 defines a first head means 701; the first sidewall 703 tail end 18. The first head 701 couples to a third sidewall 705 that links to the second tail 707. The second tail 707 and second head 702 are linked by a second sidewall 704. Further, the first and second head means define a common head-height, and the split or dual tail ends have a tail height that is substantially the same as the common head-height.

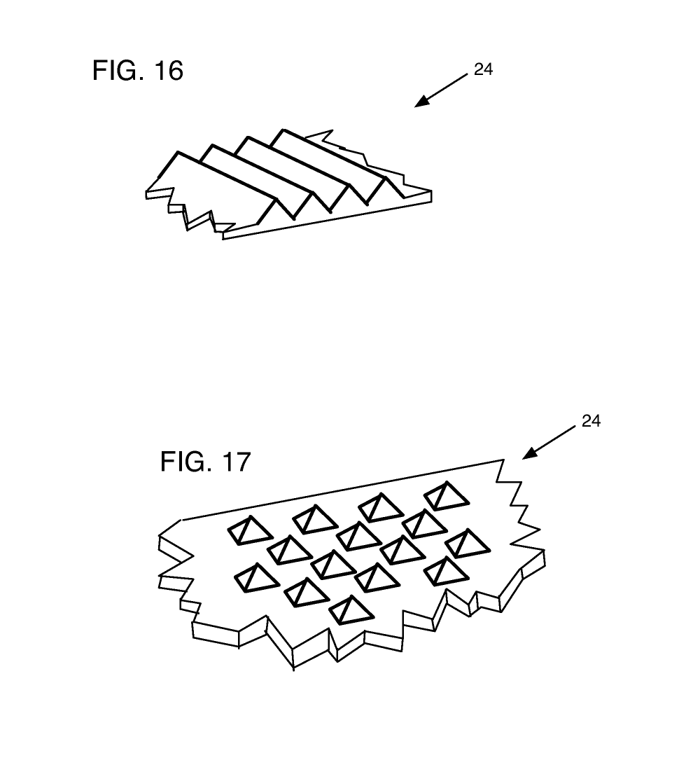

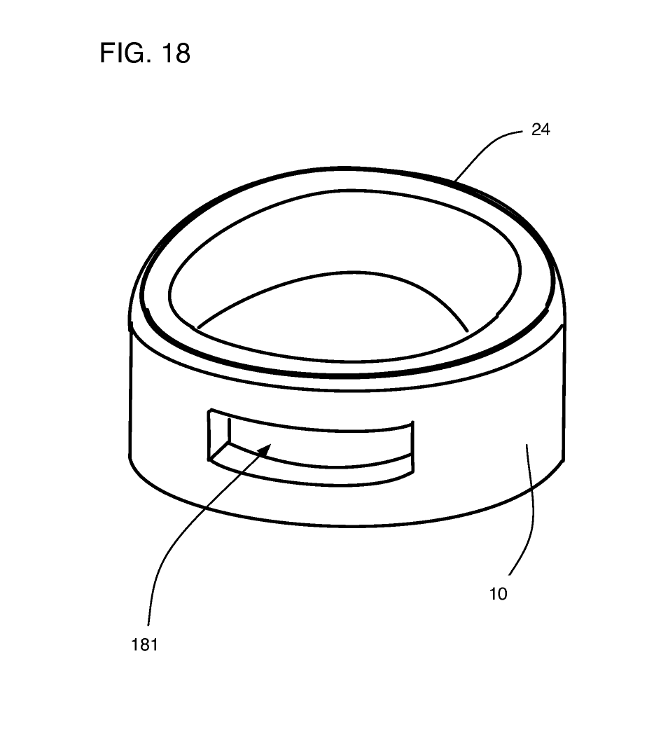

The implant 10 of FIG. 9 is essentially the same design as that of FIG. 6. This open tailed implant provides the same at least three contact points for insertion in the bone. A first contact point 101 is designed to be inserted toward the outer portion of the bone, or proximal. Two additional contact points, one on each tail portion, contact 102 and 103, provide the necessary stability when cooperating with the first contact 101 point at the head. At least one sidewall member 105 couples and links the three contact point. And, a friction enhancing feature 24 arranges on the top surface. FIGS. 16, 17, and 18 detail some contemplated friction enhancing features. For example, FIG. 16 illustrates a serrated pattern consisting of ridges having peaks and valleys that extend across a portion of the top surface, or preferably across a substantial portion of the top surface. FIG. 17 illustrates a plurality of peaked features scattered over the top surface that act like teeth. And, FIG. 18 illustrates a single ridge that arranges about the circumference of the top surface. Of course combination of these features, or permeations of these features would work equally well. And, although not specifically illustrated in the figures, the bottom surface could be void of such friction enhancing features, or preferably, include one or more of the discussed friction enhancing features or combinations of features, and the bottom friction-enhancing feature would be independent of the type of friction enhancing feature arranged on the top surface.

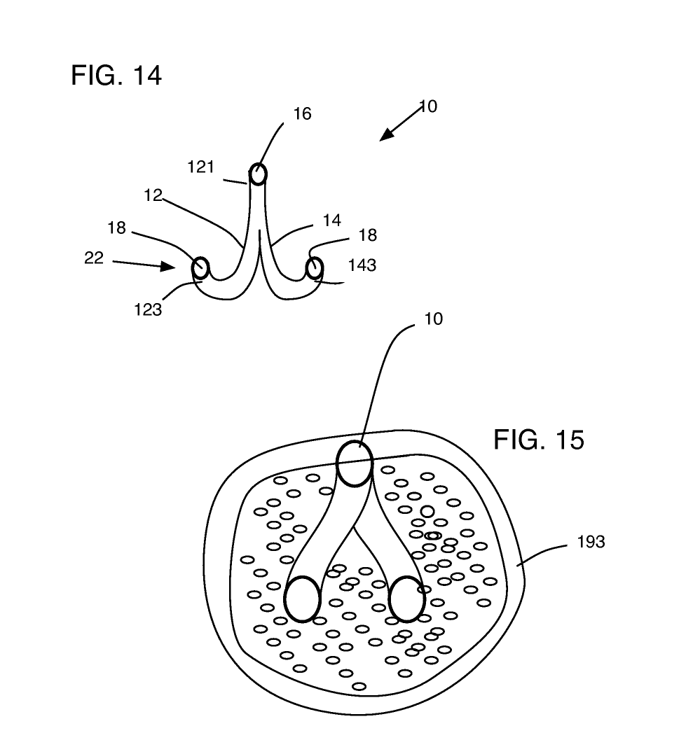

FIGS. 10, 11, 12, 13, and 14 illustrate another preferred embodiment of an implant 10 according to the present invention. Common to these designs is at least three contact points 101, 102, and 103 coupled together by body linking segments 105. And, in the case of FIG. 13, a four-point contacting implant having the fourth contact 104. Each of these embodiments may also include friction enhancing features on any at least one of the contact top surfaces, or bottom surfaces, or both, or all contact surface top and/or bottom.

FIG. 15 illustrates a contemplated method of inserting the implant 10 in a bone 193 to correct anatomical issues. And, although a three-contact implant is illustrated, any of the contemplated embodiments would work equally well.

FIG. 18 also shows a slot adapted to receive a staple, or multiple staples, one or more pins, screws, blades, tines, or rivets—as would be appreciated by those with ordinary skill in this art. Moreover, the single slot of FIG. 18 could be replaced with one or more different apertures including oval, circular, or rectilinear openings. Also contemplated are single or multiple slots, single or multiple holes or a combination; these slot(s) and or holes(s) are smooth bore, threaded bore, ratcheted bore or a combination which follow either or a combination of a straight and/or helix-corkscrew path, shaft or channel; used for fixation of the implant, insertion of the implant or a combination of both. Other openings for bone screws or pins, for example, can be included in the implant body, as would be appreciated by those skilled in the art. And a friction enhancing feature 24, such as a perimeter ring-tooth as shown, or a pair of parallel perimeter rings are also contemplated in lieu of teeth (FIG. 17) or ridges (FIG. 16).

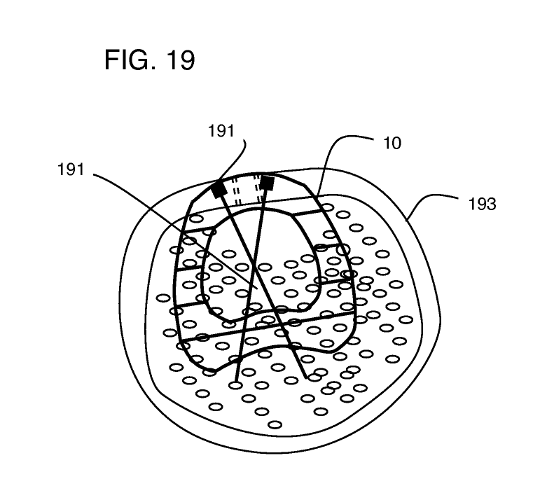

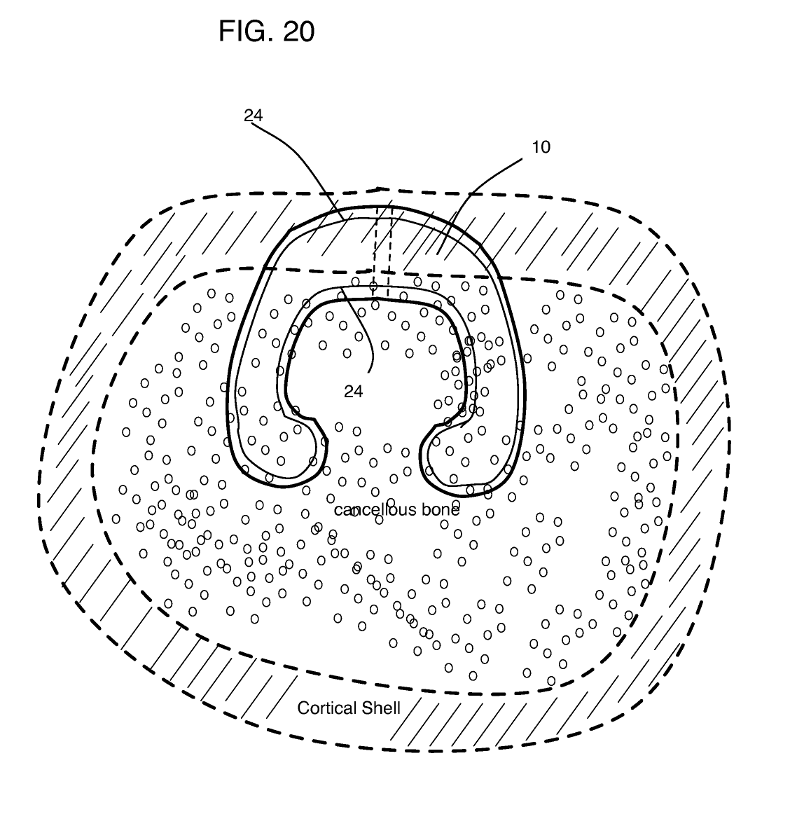

FIGS. 19 and 20 show the orientation and method of inserting the implant 10 of any of the contemplated embodiments of the present invention. Accordingly, a portion of the head lodges in the cortical shell and at least one, and preferably two contact points arrange in the cancellous bone (although the implant could be used in any bone of the foot). FIG. 19 further illustrates the use of bone screws or pins 191 with the implant device 10, as would be understood by those skilled in the art. The implant device of FIG. 20 would include a friction enhancing feature 24 including a perimeter ring as illustrated in FIG. 18, or other friction enhancing features as FIGS. 16 and 17 show, for example. Further, it will be understood by those skilled in this art that pins, C, can be used to improve fixation and location of the implant in the bone. As such they could follow divergent paths or trajectories, they may have incongruent or unequal diameter, lengths, or other dimensions such as threaded or unthreaded characteristics. And, in at least one preferred embodiment such pins (or screws, blades, tines, or rivets) utilize the one insertion point or aperture in the implant device but otherwise do not intersect or contact the implant.

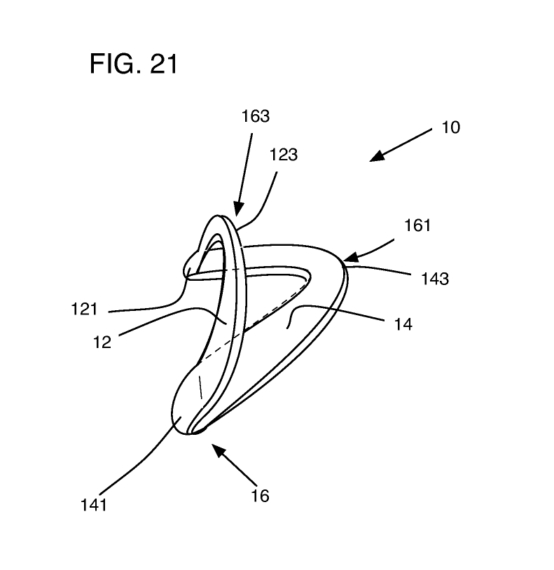

FIG. 21 illustrates yet another contemplated preferred embodiment of the implant device 10 of the present invention. As described relative to FIGS. 1-7, for example, the embodiment of FIG. 21 includes Thus, an implant body 10 comprising at least two side walls, the at least two sidewalls comprising a first sidewall 12 having a first-sidewall head-end 121 and a first-sidewall tail-end 123 and a second sidewall 14 having a second-sidewall head-end 141 and a second-sidewall tail-end 143; the implant body further comprising a head means 16 for joining at least two side walls at each respective head-end of each respective first and second sidewalls, the head having a first height 161 defined by a common height of the first-sidewall and second-side wall head-end, respectively. The implant body further having a second height 163, the second height being in a different plane from the first height.

In all the discussed contemplated embodiments of the device according to the present invention, the concept is to minimize disturbance of the cortical plate (cortex), without sacrificing or compromising sustained structural separation of cortices while healing occurs.

Although the invention has been particularly shown and described with reference to certain embodiments, it will be understood by those skilled in the art that various changes in form and detail may be made without departing from the spirit and scope of the invention.

I claim:

1. An orthopedic implant device for use to correct injuries and deformities of foot and ankle bones, the device comprising:
an implant body comprising at least two side walls, the at least two sidewalls comprising a first side wall having a first-sidewall head-end and a first-sidewall second end and a second sidewall having a second-sidewall head end and a second-sidewall tail-end;
the implant body further comprising a head means for joining at least two side walls at each respective head-end of each respective first and second sidewalls, the head having a first height defined by a common height of the first-sidewall and second-side wall head-end, respectively;
the implant body further having a second height, the second height being substantially the same as the first height, and the second height being defined by a common height of each respective first and second sidewalls at each respective tail-end;
the implant body further comprising a top portion arranged generally perpendicular to the at least two sidewalls and a bottom portion disposed opposite the top portion and being generally parallel to the top portion; and
the top portion further including a top-friction enhancing feature.

2. The implant device of claim 1 wherein:
the first sidewall comprises a first arcuate member having a first acuate radius;
the second sidewall comprises a second arcuate member having a second arcuate radius; and wherein the first and second arcuate radii are the same and wherein the first and second sidewalls cooperate to define a concave cavity therebetween.

3. The implant device of claim 2 wherein:
the head means comprises a vertical sidewall having a third arcuate radius defined by a member having a left head-end and a right head-end wherein the left head end couples to the first sidewall and the right head-end couples to the second sidewall; and wherein the first and second sidewalls and head means define a concave cavity therebetween.

4. The implant device of claim 3 further comprising:
a tail-member vertical sidewall having a fourth arcuate radius defined between the first sidewall tail-end and the second sidewall tail end, the arcuate fourth sidewall arranged to have a convex face disposed toward the concave cavity and a concave face disposed opposite its convex face.

5. The tail-member of claim 4 further comprising a though hole arranged on its concave face and extending through the tail-member sidewall terminating at the convex face.

6. The implant device of claim 1 wherein:
the friction enhancing feature of the top surface comprises at least one top-serration consisting of a top-peak disposed at a first distance from the top and the top-peak further including a first and second support member arranged to intersect at a line defined by the top-peak and terminate adjacent to the top surface, the at least one top-serration being disposed generally parallel to any adjacent serrations, the at least one top-serration further arranged to extend horizontally across the top portion wherein an axis drawn from the head to the tail would define a vertical line; and

7. The implant device of claim 6 wherein:
the top-friction enhancing feature comprising of at least one top-serration further comprises a plurality of top-serrations disposed at a pre-determined spacing peak-to-peak.

8. The implant device of claim 1 further comprising:
the bottom portion further comprising a bottom-friction enhancing feature comprising of at least one bottom-serration consisting of a bottom-peak disposed at a first distance from the bottom and the bottom-peak further including a first and second support member arranged to intersect at a line defined by the bottom-peak and terminate adjacent to the bottom surface, the at least one bottom-serration being disposed generally parallel to any adjacent serrations, the at least one bottom-serration further arranged to extend horizontally across the bottom portion.

9. The implant device of claim 8 wherein:
the bottom-friction enhancing feature comprising of at least one bottom-serration further comprises a plurality of bottom-serrations disposed opposite to and at the same a pre-determined spacing peak-to-peak as the plurality of top-serrations.

10. The implant device of claim 1 wherein:
the first sidewall comprises a first substantially straight and vertical member;
the second sidewall comprises a second substantially straight and vertical member;
and the head means comprises an intersecting line where the first sidewall head-end couples to the second sidewall head-end.

11. The implant device of claim 2 wherein:
the head means comprises a substantially flat and vertical sidewall having a left head-end and a right head-end wherein the left head end couples to the first sidewall and the right head-end couples to the second sidewall; and wherein the first and second sidewalls and head means define a concave cavity therebetween.

12. The implant device of claim 1 wherein:
the at least two sidewalls comprise three sidewalls, the three sidewalls arranged in a N-formation wherein the head comprises a first head end and a second head end, the first head end linked to a first tail end by means of a first sidewall, the first head end linked to a second tail end by means of a third sidewall, the second head end linked to the second tail end by means of a second sidewall, the third sidewall 705 linking the two outside sidewalls diagonally, and the three sidewalls arrange in a N-formation.

13. The implant device of claim 1 wherein:
the friction enhancing feature of the top surface comprises at least one ridge extending at least partially parallel to a sidewall supporting the top surface.

14. The implant device of claim 1 wherein:
the friction enhancing feature of the top surface comprises at least one ridge disposed on the top surface extending circumferentially thereon and extending parallel to at least one sidewall supporting the top surface.

15. The implant device of claim 1 wherein:
the friction enhancing feature of the top surface comprises a plurality of individual peaked features resembling a triangle when viewed in profile.

16. A method of performing a foot or ankle implant surgery wherein a specific bond of the foot or ankle is preselected for implant surgery, the method comprising:
providing an implant comprising
an implant body comprising at least two side walls, the at least two sidewalls comprising a first side wall having a first-sidewall head-end and a first-sidewall second end and a second sidewall having a second-sidewall head end and a second-sidewall tail-end;
the implant body further comprising a head means for joining at least two side walls at each respective head-end of each respective first and second sidewalls, the head having a first height defined by a common height of the first-sidewall and second-side wall head-end, respectively;
the implant body further having a second height, the second height being substantially the same as the first height, and the second height being defined by a common height of each respective first and second sidewalls at each respective tail-end;
the implant body further comprising a top portion arranged generally perpendicular to the at least two sidewalls and a bottom portion disposed opposite the top portion and being generally parallel to the top portion; and
the top portion further including a top-friction enhancing feature.
